(19) United States
(12) Patent Application Publication
(10) Pub. No.: US 2019/0203192 A1
(43) Pub. Date: Jul. 4, 2019

(54) KLK6-MEDIATED CNS-SPECIFIC ANTIBODY PRODRUG ACTIVATION

(71) Applicant: Development Center for Biotechnology, New Taipei City (TW)

(72) Inventors: Jei-Hwa YU, New Taipei City (TW); Iok-U FONG, Macau (MO); Su-Yi HSU, New Taipei City (TW); Chih-Yung HU, New Taipei City (TW); Chia-Cheng WU, New Taipei City (TW)

(73) Assignee: Development Center for Biotechnology, New Taipei City (TW)

(21) Appl. No.: 15/859,500

(22) Filed: Dec. 30, 2017

Related U.S. Application Data
(63) Continuation of application No. PCT/US2017/069135, filed on Dec. 29, 2017.

(51) Int. Cl. C12N 9/64; C07K 16/22; C07K 14/575; C07K 16/28; A61P 25/00; A61P 35/00; A61P 37/02; A61P 31/00

(52) U.S. Cl. CPC C12N 9/6445; C07K 16/22; C07K 14/5759; C07K 16/2803; A61K 2039/505; A61P 35/00; A61P 37/02; A61P 31/00; C07K 2319/30; A61P 25/00

(57) ABSTRACT

An antibody prodrug capable of being selectively activated in a central nervous system (CNS) by protease KLK6 includes an antibody for treating a disease or disorder in the CNS; a KLK6 cleavable peptide fused to an N-terminus of a heavy chain and/or a light chain of the antibody; and a blocker fused to an N-terminus of the KLK6 cleavable peptide. The disease or disorder is a cancer, inflammatory disease, autoimmune disease, infectious disease, or neuron degeneration disease.

### CROSS REFERENCE TO RELATED APPLICATIONS

This claims the benefit of PCT/US2017/069135, filed on Dec. 29, 2017, which claims the benefit of Provisional Application No. 62/440,296, filed on Dec. 29, 2016. The disclosures of these prior application are incorporated by reference in their entirety.

### BACKGROUND OF INVENTION

### Field of the Invention

The present invention relates generally to antibody-based molecules for treating various CNS-related diseases.

### Background Art

Therapy is often associated with adverse effects. To minimize the adverse effects, one needs to improve the specificities of the therapeutic agents, i.e., attacking only the disease agents or cells/tissues. However, having such selectivity is not always possible. For example, antibodies are highly specific to their target antigens with high affinities, and have been approved for therapeutic or diagnostic applications in some diseases. However, the target antigen expression is not necessarily disease-specific. Therefore, most therapeutic antibodies may also target normal tissues in addition to targeting the disease lesions, thereby causing adverse effects.

Another approach to reducing the adverse effects is to deliver the therapeutic agents as pro-drugs, which are activated only at the target sites. Many prodrugs are shown to be effective in reducing the undesired side effects. However, most prodrugs are small molecules.

Central nervous system (CNS) presents unique challenges for specific targeting of therapeutic agents. Therefore, there is still a need for better approaches to the treatment of CNS diseases or disorders.

### SUMMARY OF INVENTION

Embodiments of the present invention relate to uses of KLK6 substrate-containing recombinant protein or peptide to fuse with the N-termini of the antibody to produce prodrug antibodies (pro-antibodies) with reduced antigen recognition function. These pro-antibodies can be activated in tissues having high concentrations of KLK6 protease, such as in the CNS.

Inventors of the present invention found that the KLK6 substrate sequences in the pro-antibodies are not cleaved by the KLK6 in serum due to the low concentration of KLK6 in serum. In contrast, the pro-antibodies are cleaved by the KLK6 in the CNS due to higher concentrations of KLK6 therein. Therefore, the functions of the antibodies can be suppressed in the peripheral blood to prevent the adverse effects and to minimize the waste of antibodies in the peripheral tissues. These pro-antibodies can be activated to target CNS-related diseases or disorders once it gets into the CNS.

In one aspect, embodiments of the invention relate to antibody prodrugs capable of being selectively activated in a central nervous system (CNS) by protease KLK6. An antibody prodrug in accordance with one embodiment of the invention includes an antibody, or a binding fragment thereof, for treating a disease or disorder in the CNS; a KLK6 cleavable peptide fused to an N-terminus of a heavy chain and/or a light chain of the antibody; and a blocker fused to an N-terminus of the KLK6 cleavable peptide.

In accordance with embodiments of the invention, the disease or disorder in the central nervous system (CNS) may be a cancer, inflammatory disease, autoimmune disease, infectious disease, or neuron degeneration disease.

In accordance with embodiments of the invention, the blocker comprises a protein, protein domain, or a peptide. In accordance with embodiments of the invention, the KLK6 cleavable peptide is derived from a natural KLK6 substrate, a peptide phage library, a synthetic peptide library, or any peptide sequence that can be cleaved by KLK6.

In accordance with embodiments of the invention, the antibody is a monoclonal antibody or a binding fragment thereof, bispecific antibody, multi-specific antibody, antibody drug conjugates (ADC), immunocytokine, immunohormone, or immunotoxin. The antibody may be a murine antibody, chimeric antibody, humanized antibody, or human antibody. In accordance with embodiments of the invention, the antibody may be any suitable antibody, such as anti-VEGF antibody or anti-PD-1 antibody

In another aspect of the invention, embodiments of the invention relate to methods for treating a disease or disorder in the CNS. A method in accordance with one embodiment of the invention comprises: administering to a subject in need thereof an effective amount of an antibody prodrug (i.e., pro-antibody) of the invention.

An effective amount is the amount that can achieve the desired effects of the treatment. One skilled in the art would appreciate that the effective amount would depend on the disease conditions, the route of administration, the conditions of the patient (e.g., age, body weight, gender, etc.), and one skilled in the art would be able to determine the effective amount without undue experimentation. For example, in accordance with embodiments of the invention, an effective amount may range from 0.1 μg/Kg to 10 mg/Kg, preferably from 1 μg/Kg to 1 mg/Kg, more preferably from 1 μg/Kg to 0.1 mg/Kg, based on the weight of the patient.

Other aspects and advantages of the invention will be apparent from the following description and the appended claims.

### BRIEF DESCRIPTION OF THE DRAWINGS

FIG. 1 shows a diagram illustrating a strategy of KLK6-mediated CNS-specific antibody prodrug action.

FIG. 2(A) shows results of in vitro KLK6 cleavage of KLK6 substrate (KLK6s)-containing protein-fused antibody Fab by Coomassie staining of the protein bands. FIG. 2(B) shows Western blot.

FIG. 3(A)-FIG. 3(C) show the antigen recognition ability of a KLK6 substrate (KLK6s)-containing protein-fused antibody Fab, before and after KLK6 digestion. FIG. 3(A) shows binding of anti-VEGF Fab (Lucentics®) control as measured with surface plasmon resonance (BIAcore). FIG. 3(B) shows that pro-antibody could not bind the antigen. FIG. 3(C) shows that after KLK6 digestion, the pro-antibody was activated to reveal binding to the antigen.

FIG. 4 shows results of in vitro KLK6 cleavage of KLK6 substrate (KLK6s)-containing peptide fused antibody.

FIG. 5(A)-FIG. 5(E) show results of antigen recognition ability of the KLK6 substrate (KLK6s)-containing peptide fused antibody, before and after KLK6 digestion. FIG. 5(A) shows binding of the Avastin control. FIG. 5(B) shows that the FR1 peptide-KLK6s fused Avastin-KLK6s-Leptin without KLK6 digestion exhibited reduced binding affinity to its antigen VEGF (at 0 hr of KLK6 digestion). With increasing KLK6 digestion time, the FR1 peptide-KLK6s fused Avastin-KLK6s-Leptin was progressively activated for VEGF binding (6, 24, and 48 hrs in FIGS. 5(C), 5(D), and 5(E), respectively).

FIG. 6(A) and FIG. 6(B) show results of in vitro KLK6 cleavage as revealed by CBR staining, and FIG. 6(C) shows results of antigen recognition ability before and after KLK6 digestion of the Avastin prodrugs with different peptide blockers and KLK6 substrates.

FIG. 7(A) shows results of in vitro KLK6 cleavage, and FIG. 7(B) shows antigen recognition ability before and after KLK6 digestion of anti-PD-1 mAb Opdivo prodrugs.

FIG. 8 shows results of in vivo antibody activation of the KLK6 substrate (KLK6s)-containing peptide-fused antibody in mice.

### DEFINITIONS

As used herein, the term “antibody” refers to a monoclonal antibody, a bispecific antibody, a multi-specific antibody, or an antibody drug conjugate (ADC). The “antibody drug conjugate” may be an immunocytokine, an immunohormone, or an immunotoxin. The term “immunocytokine” refers to a recombinant antibody-cytokine fusion protein. The term “immunohormone” refers to a recombinant antibody-hormone fusion protein. An example of an immunohormone is an antibody-leptin fusion protein. The term “immunotoxin” refers to a recombinant antibody-toxin conjugate. A binding fragment of an antibody may be an scFv, Fab, or F(ab′)₂.

The term “fused to an N-terminus of a protein” refers to forming a fusion protein at the N-terminus of the protein. The term “blocker” refers to a peptide or protein used to reduce or block the activity of an antibody, i.e., to convert the antibody into a prodrug. In addition, the blocker may be used to facilitate delivery into the CNS system, see e.g., Yu, Y. J. et al., “Boosting brain uptake of a therapeutic antibody by reducing its affinity for a transcytosis target,” Sci. Trans. Med. 3, 84ra44 (2011).

The term “KLK6 cleavable peptide” refers to a peptide that can be cleaved by KLK6, which is a trypsin-like protease. Various KLK6 cleavable peptide sequences are known in the art or one skilled in the art can easily determine whether a particular sequence can be cleaved by KLK6 without undue experimentation.

### DETAILED DESCRIPTION

Embodiments of the invention relate to therapeutic antibodies for the treatments of diseases or disorders in the central nervous system (CNS). These antibodies are in the form of a prodrug (pro-antibody), which includes a peptide fragment (blocking peptide, or blocker) attached to the N-terminus of a variable domain of an antibody to block the binding of the antibody to the target antigen. The blocking peptides are similar to the pro-peptides of certain proenzymes. Cleavage of the blocking peptides by a selective protease will release the active antibodies.

Embodiments of the invention rely on kallikrein-6 (KLK-6) for the specific activation of the pro-antibodies. Specifically, embodiments of the present invention use KLK6 substrate-containing recombinant protein or peptide to fuse with the N-termini of an antibody. The KLK6 substrate-containing recombinant protein or peptide functions like a pro peptide to block or reduce the antigen recognition function of the antibody.

The KLK6 substrate would not be cleaved to any appreciable extent by the KLK6 in blood circulation due to the low concentrations of KLK6 in the serum in peripheral blood. However, the KLK6 substrate-containing recombinant protein or peptide can be efficiently cleaved by the KLK6 at a concentration found in the CNS. Therefore, the function of the antibodies can be blocked or minimized in the peripheral blood to prevent the adverse effects and to reduce the unnecessary waste of the antibodies. These pro-antibodies can be activated to target CNS-related diseases once it gets into the CNS.

The human kallikrein (KLK) family includes 15 secreted serine proteases that are expressed in various tissues. Among these, KLK6 is found to be highly expressed in CNS and is also overexpressed in breast and ovarian cancer tissues. KLK6, also known as zyme, protease M, and neurosin, is a trypsin-like serine protease. KLK6 is constitutively expressed in oligodendrocytes in the CNS and its expression is enhanced after spinal cord injury (SCI) and in active multiple sclerosis (MS) lesions. Many proteins have been shown to be cleaved by KLK6, including human myelin basic protein (MBP), beta amyloid peptide (Aβ), plasminogen, myelin, and α-synuclein, etc.

The KLK6 levels in various human fluids have been determined, and KLK6 has been found to be significantly enriched in the cerebrospinal fluid (CSF), as compared to blood circulation. This differential expression levels enable an opportunity for pro-drug activation in the CNS.

In accordance with embodiments of the invention, to reduce the unwanted side effects, antibody prodrugs with reduced function in the peripheral blood have been developed. These antibody prodrugs can be activated by KLK6 in the central nervous system. The invention can be applied in various CNS-related diseases, e.g., cancers, inflammatory diseases, autoimmune diseases, infectious diseases, or neuron degeneration diseases.

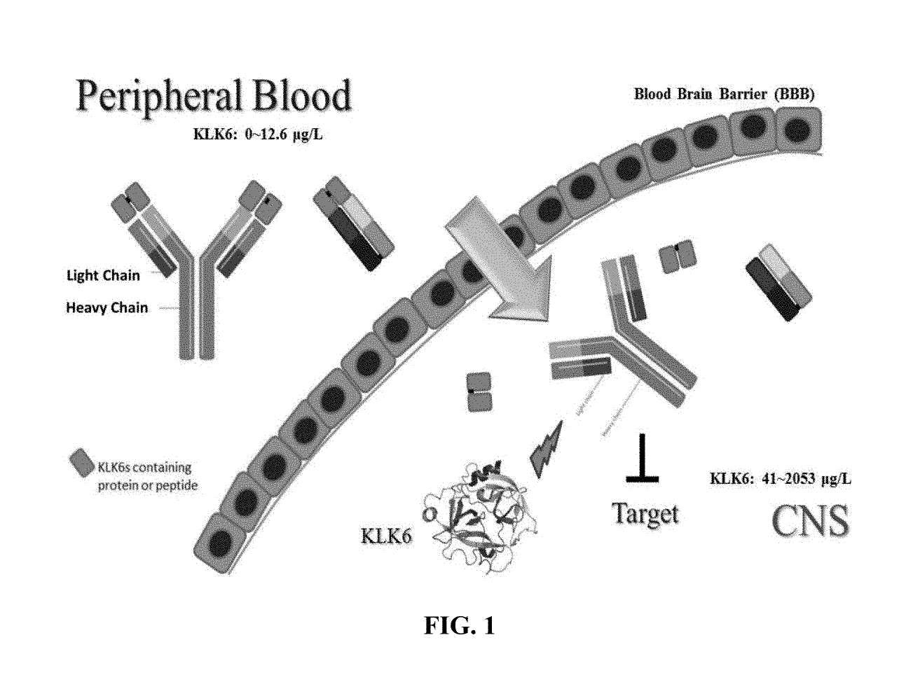

FIG. 1 shows a diagram illustrating a strategy of KLK6-mediated CNS-specific antibody prodrug action. In accordance with embodiments of the invention, the N-termini of the light chain and/or heavy chain of an antibody may be fused with a blocker (blocking peptide) to convert the antibody into a prodrug (i.e., pro-antibody). The blocker reduces the antigen recognition of the antibody. The blocker contains a peptide sequence that can be cleaved by KLK6. The peptide sequence functions as a KLK6 substrate (KLK6s).

In accordance with some embodiments of the invention, only the light-chain N-terminus contains a blocker. In accordance with other embodiments of the invention, only the heavy-chain N-terminus contains a blocker. Because antigen-antibody binding involves the variable domains of both the heavy-chain and the light-chain, blocking either the heavy-chain or the light-chain N-terminus would be sufficient to disrupt the antigen-antibody binding.

In accordance with some embodiments of the invention, both the light-chain N-terminus and the heavy-chain N-terminus each contain a blocker. The dual blocking of both the heavy-chain and the light-chain variable regions may produce better blocking of the antigen-antibody interactions.

In accordance with embodiments of the invention, a blocker comprises a KLK6 substrate-containing protein, protein domain, or peptide, such that the active antibody will be released only in the environment having KLK6 above certain concentration. KLK6 has been shown to be a trypsin-like protease, preferring to cleave on the carboxyl side of an arginine residue. Li et al. found that KLK6 prefers a peptide substrate with the P1 position occupied by Arg and a strong preference for Ser in P1′. (Protein Sci., 17(4): 664-672 (2008)). Magklalra et al. (Biochem. Biophys. Res. Commun., 307(4): 948-55 (2003)) also found that KLK6 cleaves with much higher efficiency after Arg than Lys and with a preference for Ser or Pro in the P2 position. Based on this information, one can design a peptide sequence that would be efficiently cleaved by KLK6. One skilled in the art would appreciate that many peptide sequences would fulfill these criteria and embodiments of the invention are not limited to any particular examples used in this description. Such a peptide will be referred to generically as a KLK6 substrate (or KLK6s) in this description.

Embodiments of the invention are based on the observation that KLK6 exists in a much higher concentration in the CNS than in the peripheral blood. The concentration of KLK6 in the peripheral blood ranges from 0 to 12.6 μg/L, whereas the concentration of KLK6 in the CNS ranges from 41 to 2053 μg/L. With this significant difference in the KLK6 concentrations between the peripheral blood and CNS, a blocker fusion antibody (pro-antibody) of the invention can maintain its low activity in the peripheral blood to prevent any adverse effects, but can be activated by the KLK6 in the CNS for its therapeutic function.

The following description uses specific examples to illustrate embodiments of the invention. One skilled in the art would appreciate that these examples are for illustration only and are not intended to limit the scope of the invention because variations and modifications are possible without departing from the scope of the invention.

In the following examples, various fusion proteins are prepared by constructing expression vectors containing the desired sequences. Any suitable vectors known in the art may be used. In the following examples, the KLK6s-containing peptide/protein-fused antibodies were constructed by polymerase chain reaction (PCR) and cloned into the multiple cloning sites of pTCAE8 vector (plasmid). The particular cDNA sequences for these proteins and the vectors are known in the art and readily available. The methods used are conventional. The various vectors and reagents may be obtained from commercial sources. Thus, one skilled in the art would be able to obtain these reagents and perform the described experiments without undue experimentation.

### EXAMPLE 1

### Specific Cleavage of Block Proteins from a Pro-Antibody

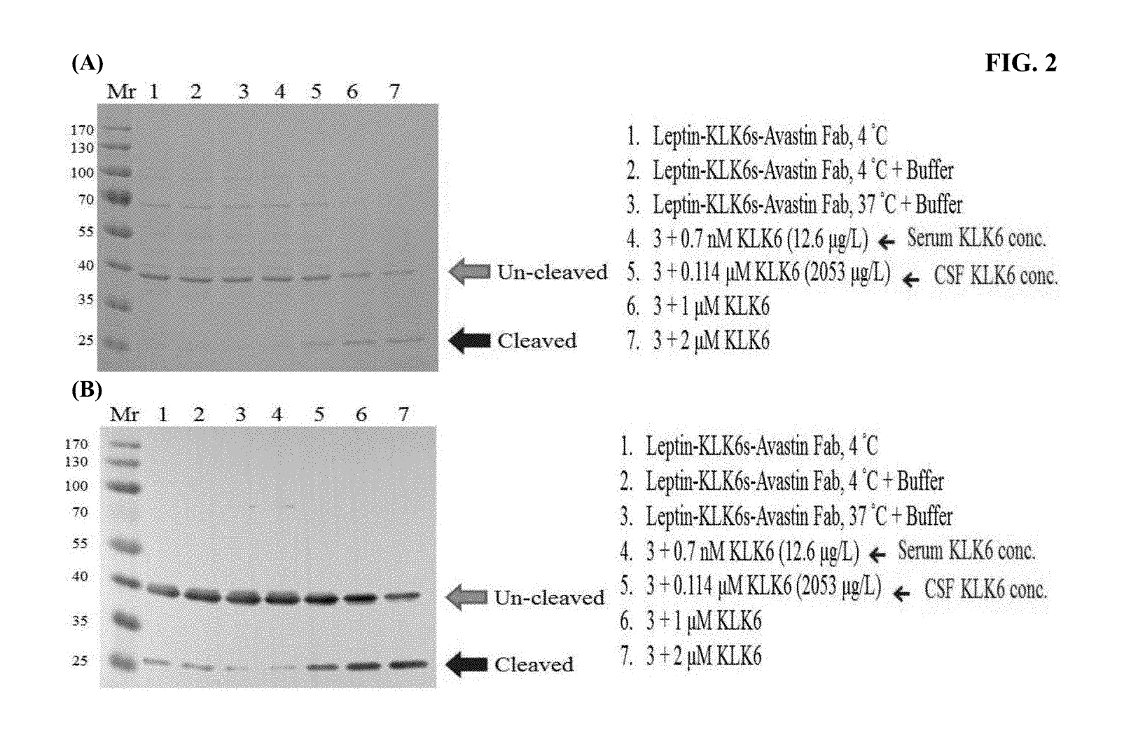

In this example, a leptin-KLK6s fused Avastin Fab is used as a pro-antibody to test the ability of KLK6 to release Avastin Fab. The fusion protein (pro-antibody) is incubated with KLK6 at the serum concentration, the CSF concentration, 1 μM, or 2 μM, respectively, for 24 hours. FIG. 2 shows results of in vitro KLK6 cleavage of KLK6 substrate (KLK6s)-containing protein-fused antibody Fab. In this example, the KLK6s (KLK6 substrate sequence) is an octapeptide having the sequence of YMTRSAMG (SEQ ID NO: 1), which contains the favorable KLK6 cleavage site -R-S-.

The proteins were separated by SDS-PAGE and stained with Coomassie Brilliant Blue R-250 (CBR) (FIG. 2A) or analyzed by western blot (FIG. 2B). Compared to the control (Lanes 1, 2, and 3), KLK6 at the serum concentration was not efficient in cleaving the blocker (Lane 4), whereas KLK6 at the CSF concentration or higher showed efficient cleavages, as evidenced by the cleaved protein indicated by arrow (Lanes 5, 6, and 7).

These results indicate that a pro-antibody in accordance with embodiments of the invention would survive in the peripheral blood and can be efficiently activated once in the CNS.

### EXAMPLE 2

### Antigen Binding by Pro-Antibody and Reactivated Antibody

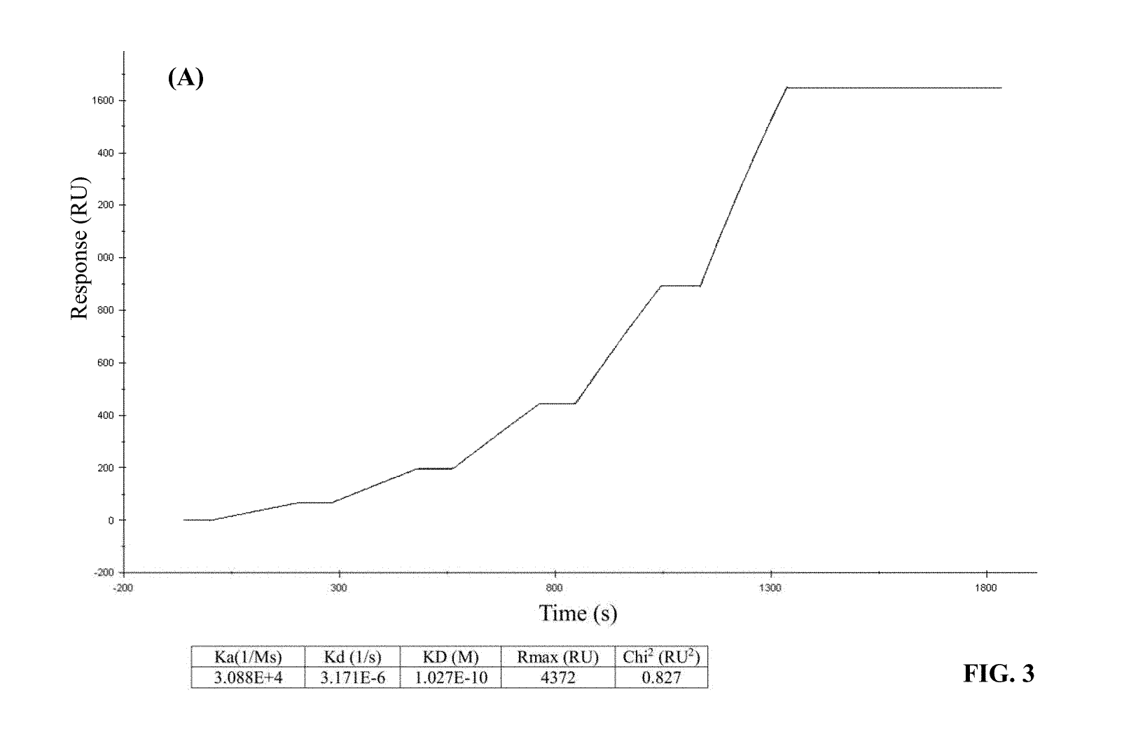

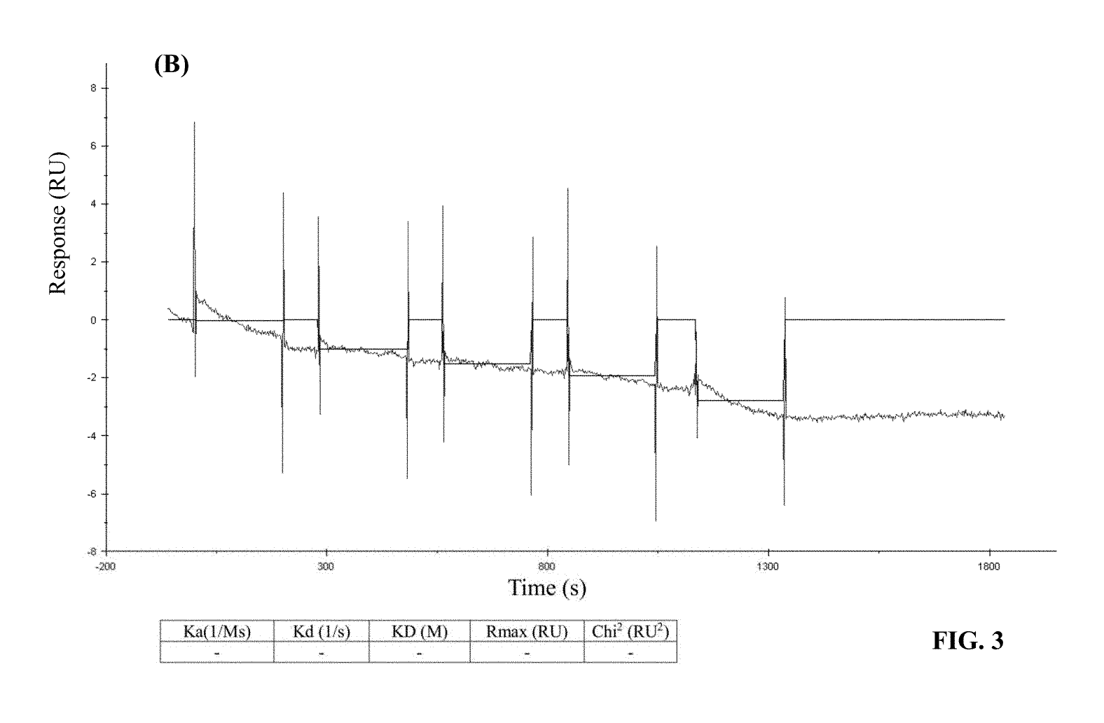

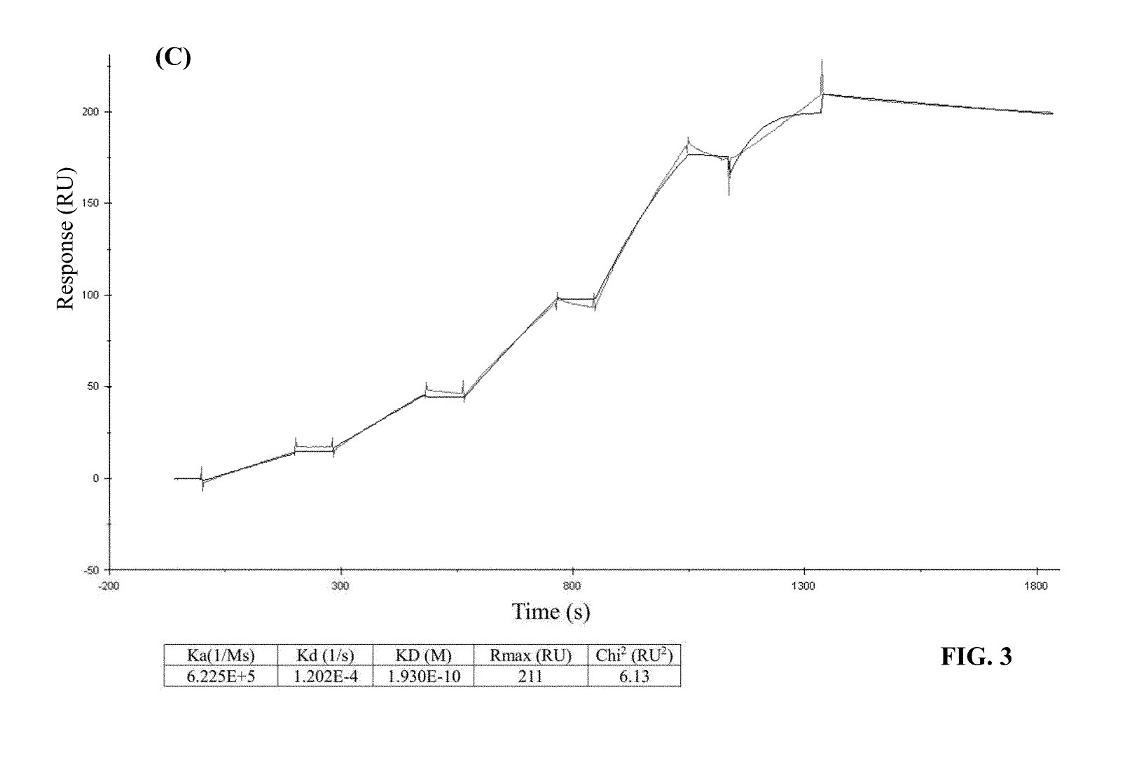

In this example, the binding kinetics of anti-VEGF Fab (Lucentics®; ranibizumab) or the Leptin-KLK6s fused Avastin Fab is determined by Surface Plasmon Resonance (SPR) (BIAcore). FIG. 3 shows the antigen recognition ability of a KLK6 substrate (KLK6s)-containing protein-fused antibody Fab, before and after KLK6 digestion. Compared to the Lucentics® control (FIG. 3A), the Leptin-KLK6s fused Avastin Fab without KLK6 digestion showed no binding affinity to its antigen VEGF (FIG. 3B). In contrast, the Leptin-KLK6s fused Avastin Fab after KLK6 digestion showed good binding affinity to VEGF (FIG. 3C).

These results show that the blocking peptide/protein was effective in preventing pro-antibody bindings to its antigen and that after cleavage of the blocker (pro-peptide or pro-protein), the active antibody was released and could bind specifically to its target. These results validate the approach of the invention.

### EXAMPLE 3

### Efficiency of KLK6 Cleavage of the Pro-Peptide

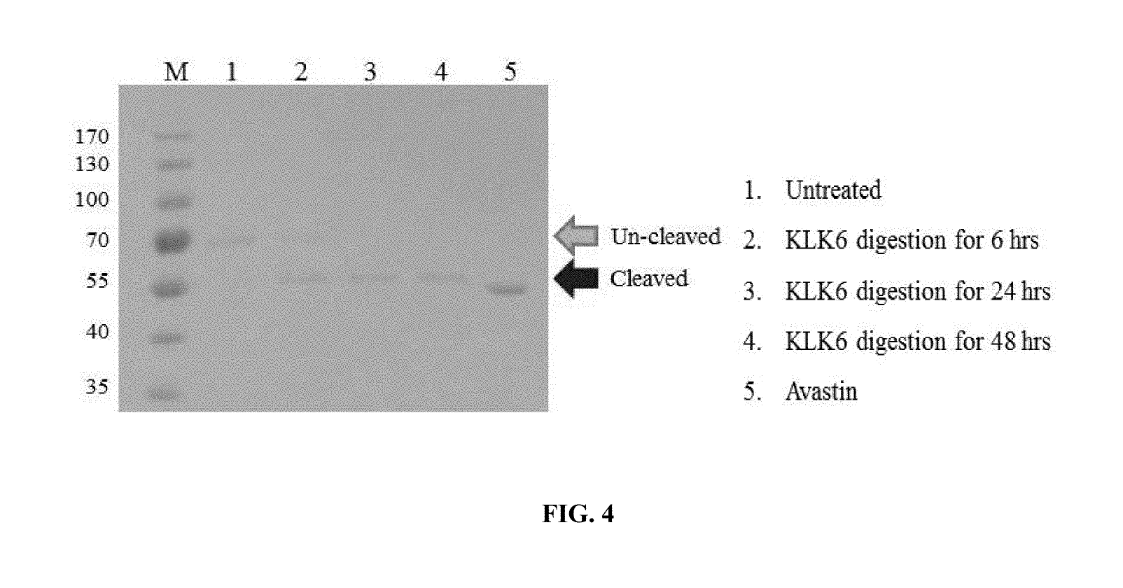

In this example, an FR1 peptide-KLK6s fused Avastin-KLK6s-Leptin was incubated with KLK6 at the CSF concentration for 6, 24, or 48 hours, respectively, wherein the FR1 peptide is the framework region 1 of Avastin. The proteins were then separated by SDS-PAGE and stained with CBR. FIG. 4 shows results of in vitro KLK6 cleavage of KLK6 substrate (KLK6s)-containing peptide fused antibody. Compared to the control (Lane 1), the KLK6 at the CSF concentration at different time point showed the cleaved protein as indicated by arrow. At 6 hours, the cleavage is substantially complete, indicating that the cleavage is quite efficient.

### EXAMPLE 4

### Time-Dependent Cleavage of the Blocker by KLK6

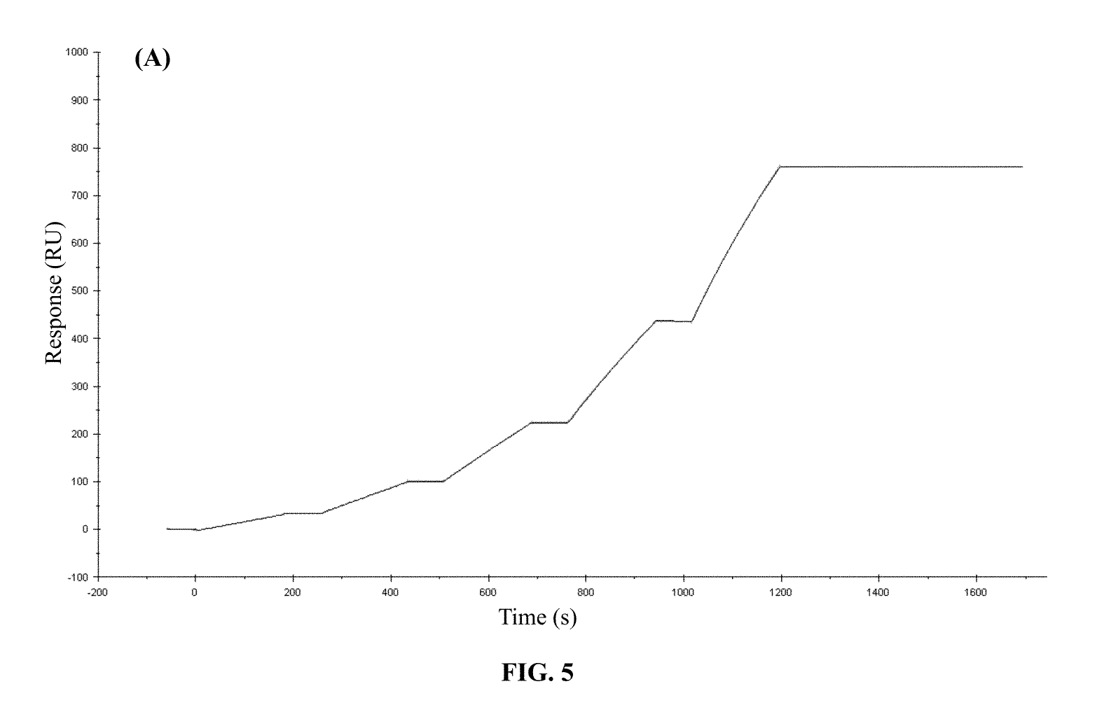

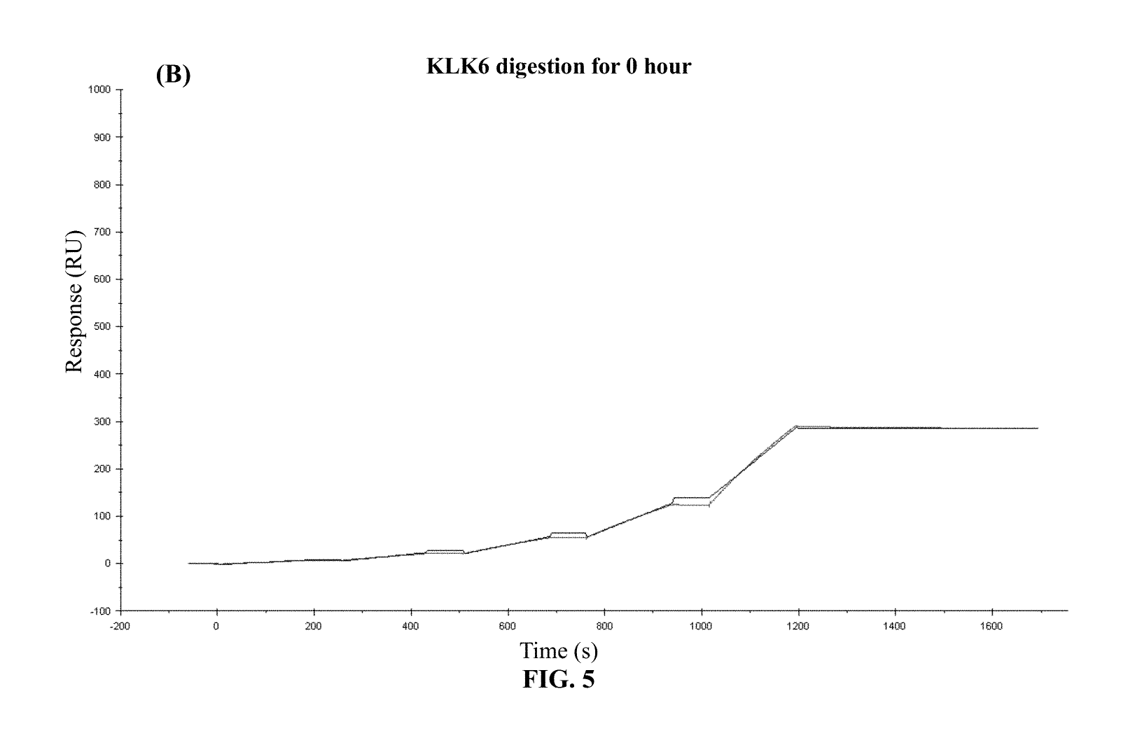

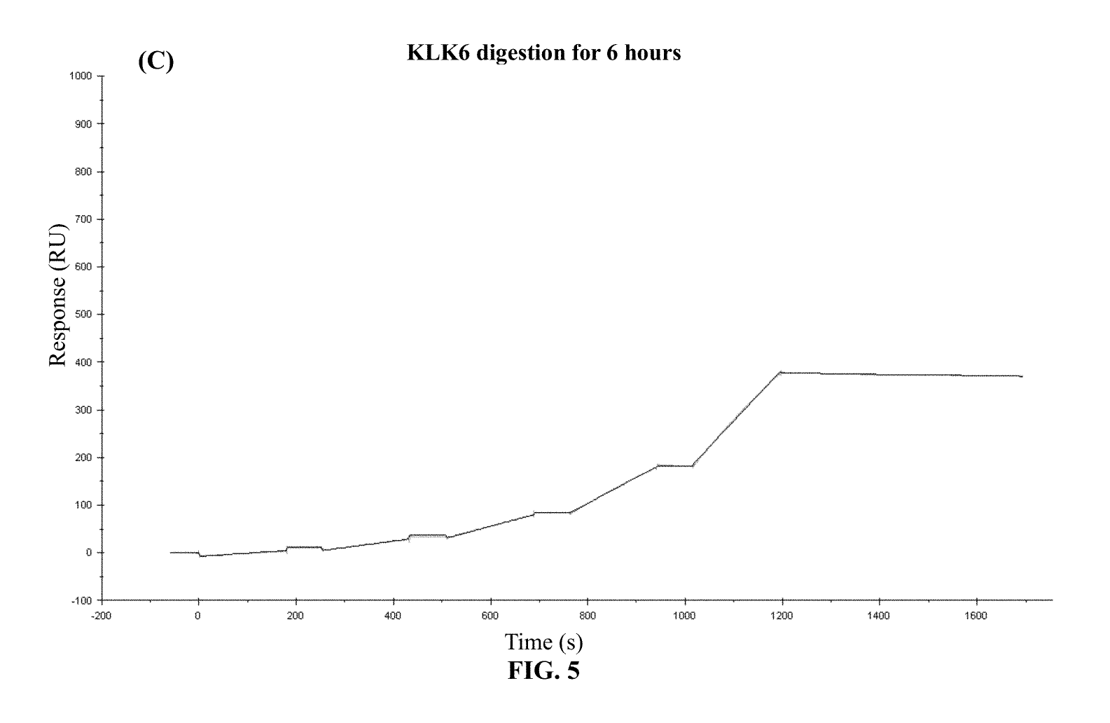

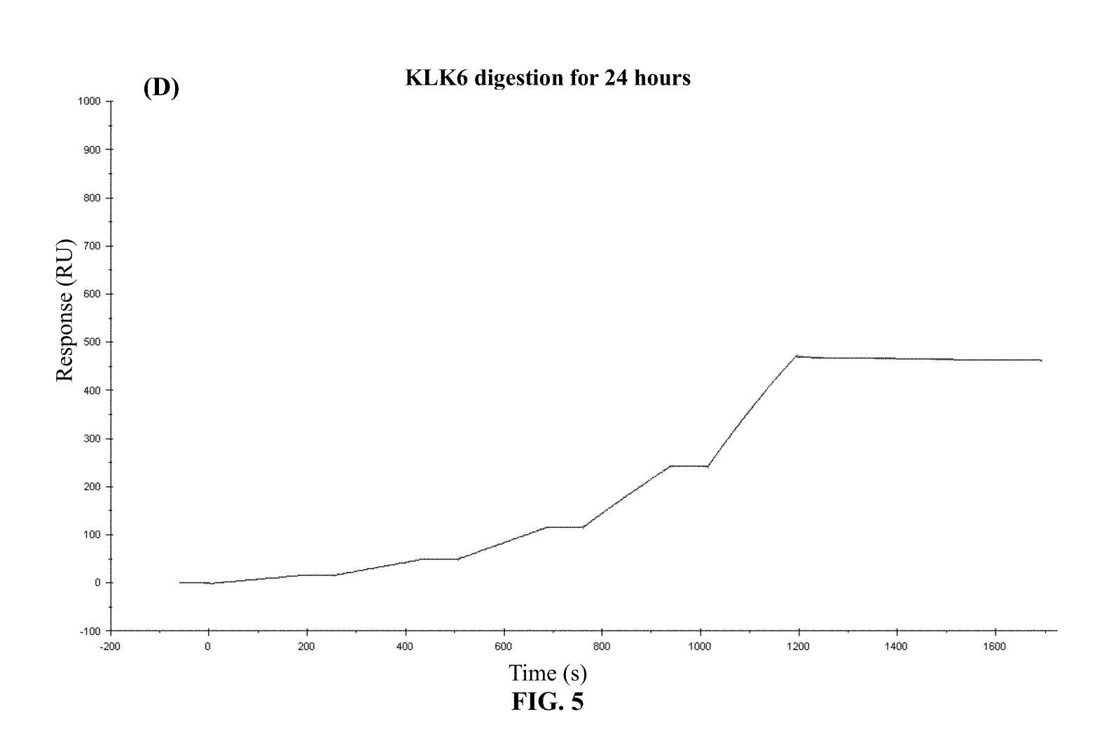

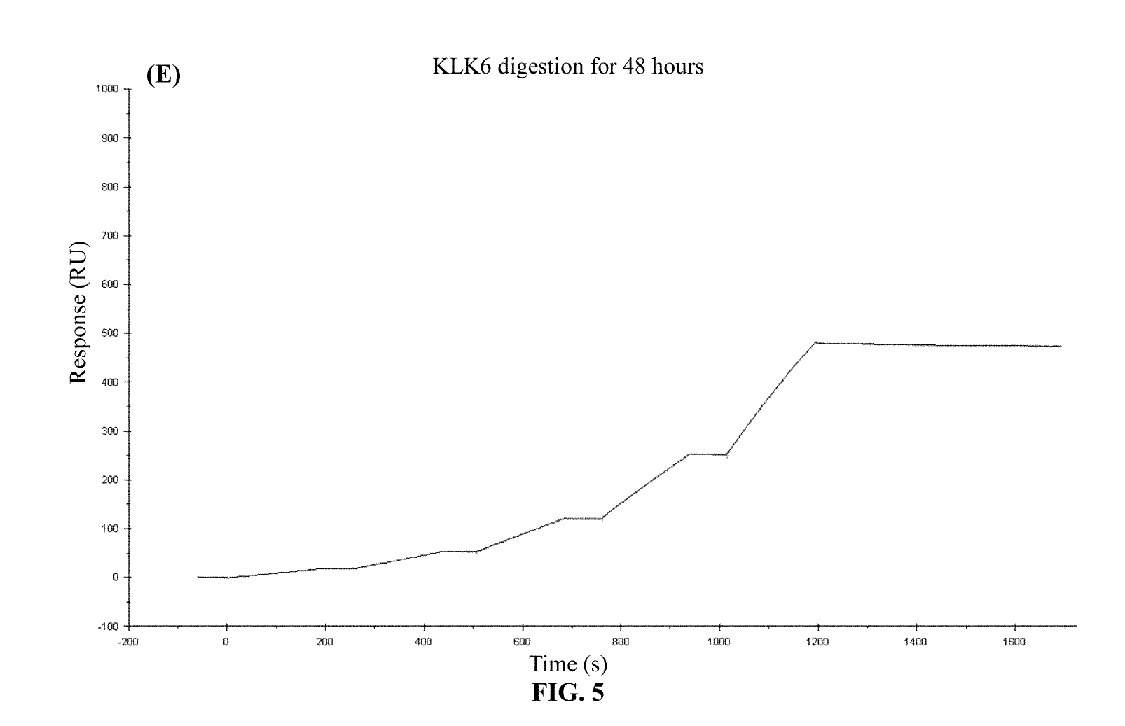

In this example, the binding kinetics of anti-VEGF antibody (Avastin) or the FR1 peptide-KLK6s fused Avastin-KLK6s-Leptin is determined by SPR (BIAcore). FIG. 5 shows results of antigen recognition ability of the KLK6 substrate (KLK6s)-containing peptide fused antibody, before and after KLK6 digestion. Compared to the Avastin control (FIG. 5A), the FR1 peptide-KLK6s fused Avastin-KLK6s-Leptin without KLK6 digestion showed reduced binding affinity to its antigen VEGF (0 hr in FIG. 5B), whereas with increasing KLK6 digestion time the FR1 peptide-KLK6s fused Avastin-KLK6s-Leptin was reactivated for VEGF binding (6, 24, and 48 hrs in FIGS. 5C, 5D, and 5E, respectively). These results corroborate that embodiments of the invention can be activated by KLK6.

### EXAMPLE 5

### KLK6-Mediated Blocker Cleavage of Pro-Antibodies with Different Peptide Blockers and KLK6 Substrates

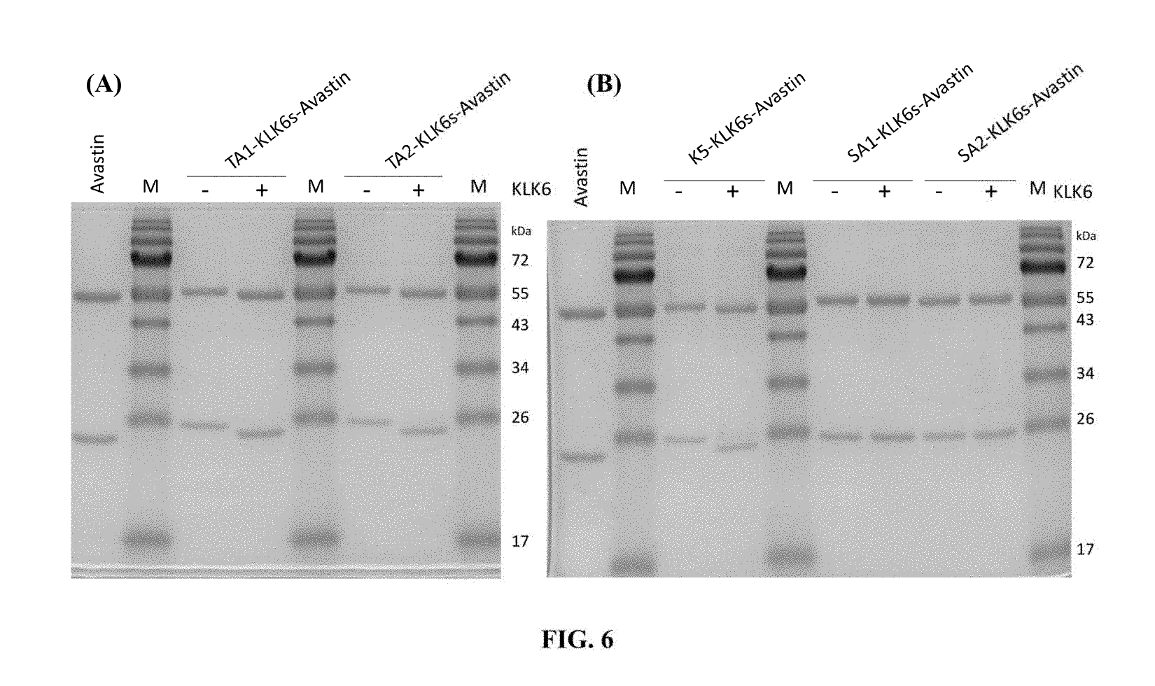

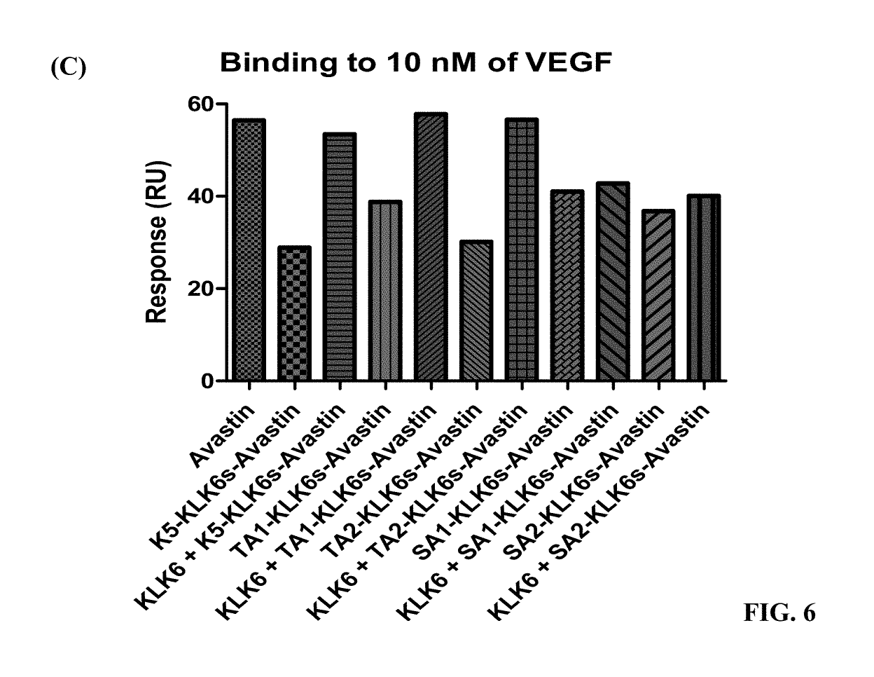

In this example, different KLK6s-containing peptide blockers fused Avastin are incubated with KLK6 at the CSF concentration for 24 hours. FIG. 6 shows results of in vitro KLK6 cleavages of KLK6s-containing peptide-fused Avastin, as analyzed by SDS-PAGE and stained with CBR (FIGS. 6A and 6B). All KLK6-cleavable peptide containing blockers fused Avastin can be cleaved by KLK6. The interaction between VEGF and the Avastin or Avastin derivatives before and after KLK6 digestion are determined by SPR (BIAcore) (FIG. 6C).

As compared with the Avastin control, different KLK6-cleavable peptide containing blockers fused Avastin without KLK6 digestion showed reduced responses, whereas with the KLK6 digestion the different KLK6-cleavable peptide containing blockers fused Avastin was reactivated for VEGF binding. In the peptide blockers fused Avastin with the uncleavable linker sequence (SSYISNYG; SEQ ID NO:13), the VEGF binding ability of the peptide blockers fused Avastin showed reduced response units before and after KLK6 digestion. These results demonstrated that embodiments of the invention with different blocker sequences or KLK6 substrate sequences can be activated by KLK6. In this example, the peptide blocker sequences and the KLK6s sequences are listed in Table 1. As shown, the peptide blocker sequences may include certain amino acids that function as spacers to separate the KLK6 substrate sequences from the antibody sequences. The spacer function to ensure that KLK6 can access the cleavage sequences. The peptide blocker sequences to the N-terminal side of the cleavage sequences can be any protein or any peptide sequences.

TABLE 1

| Name | Chain | Peptide Blocker Sequence + KLK6s (SEQ ID) | KLK6 Sequence (SEQ ID) | Cleaved |
|---|---|---|---|---|
| K5-KLK6s- | LC | DIQMGRQSCGGFGFGYMTRSAMGGGG (2) | YMTRSAMG (1) | Y |
| Avastin | HC | EVQLVESCGGFGFGYMTRSAMGGGG (3) | YMTRSAMG (1) | Y |
| TA1-KLK6s- | LC | DIQMGRQSCGGGGGGTAFRSAYGGGG (4) | TAFRSAYG (12) | Y |
| Avastin | HC | EVQLVESCGGGGGGTAFRSAYGGGG (5) | TAFRSAYG (12) | Y |
| TA2-KLK6s- | LC | DIQMGRQSCGGFGFGTAFRSAYGGGG (6) | TAFRSAYG (12) | Y |
| Avastin | HC | EVQLVESCGGFGFGTAFRSAYGGGG (7) | TAFRSAYG (12) | Y |
| SA1-KLK6s- | LC | DIQMGRQSCGGGGGGSSYISNYGGGG (8) | SSYISNYG (13) | N |
| Avastin | HC | EVQLVESCGGGGGGSSYISNYGGGG (9) | SSYISNYG (13) | N |
| SA2-KLK6s- | LC | DIQMGRQSCGGFGFGSSYISNYGGGG (10) | SSYISNYG (13) | N |
| Avastin | HC | EVQLVESCGGFGFGSSYISNYGGGG (11) | SSYISNYG (13) | N |

### EXAMPLE 6

### Specific Cleavage of Peptide Blocker from Pro-Antibodies of Opdivo®

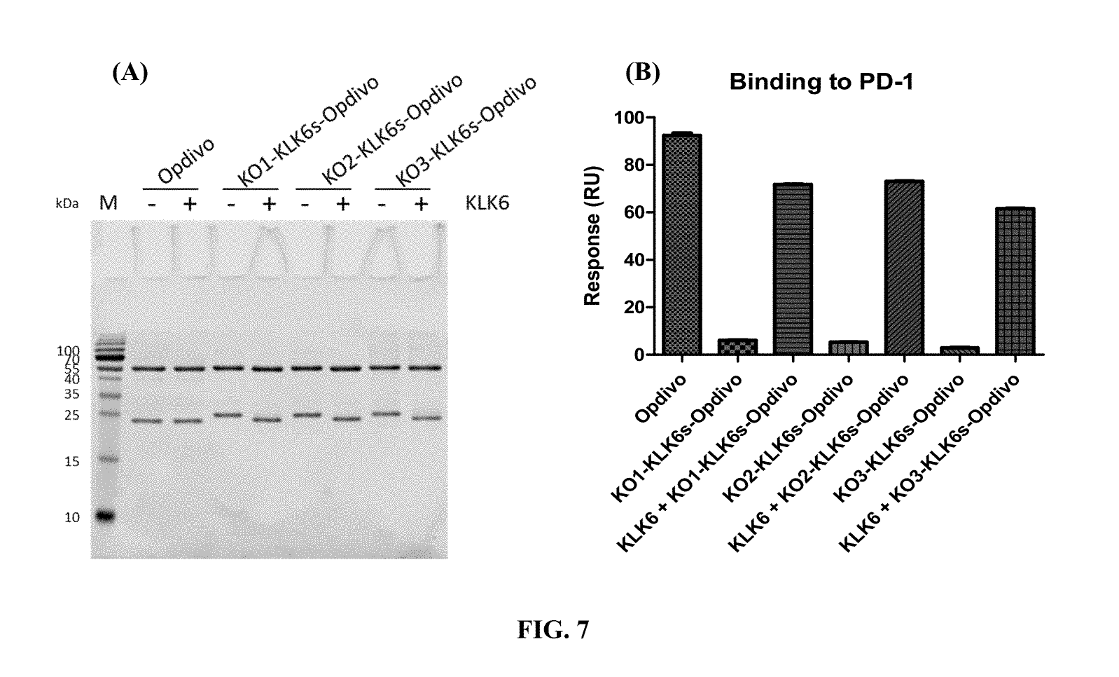

In this example, KLK6s-containing peptide blocker fused to the N-termini of anti-PD-1 mAbs (Nivolumab; Opdivo®) are incubated with KLK6 at the CSF concentration for 24 hours. FIG. 7A shows results of in vitro KLK6 cleavage of KLK6s-containing peptide-fused Opdivo® as analyzed with SDS-PAGE and stained with CBR. The KLK6s-containing peptide-fused Opdivo® can be cleaved by KLK6. The interaction between PD-1 and the anti-PD-1 mAb (Opdivo®) or Opdivo® derivatives before and after KLK6 digestion are determined by SPR (BIAcore) (FIG. 7B). As compared with the Opdivo® control, the KLK6s-containing peptide blockers fused Opdivo® without KLK6 digestion showed reduced responses, whereas with the KLK6 digestion the KLK6s-containing peptide blocker fused Opdivo® was reactivated for PD-1 binding. These results corroborate that embodiments of the invention applied in different antibody can also be activated by KLK6. In this example, the peptide blocker sequence and the KLK6s sequence are listed in Table 2.

TABLE 2

| Name | Chain | Peptide Blocker + KLK6s Sequence (SEQ ID) | KLK6 Sequence (SEQ ID) | Cleaved |
|---|---|---|---|---|
| KO1-KLK6s- | LC | EIVLTQSCGGGGGGYMTRSAMGGGG (14) | YMTRSAMG (1) | Y |
| Opdivo ® | HC | QVQLVESCGGGGGGYMTRSAMGGGG (15) | YMTRSAMG (1) | Y |
| KO2-KLK6s- | LC | EIVLTGRSCGGGGGGYMTRSAMGGGG (16) | YMTRSAMG (1) | Y |
| Opdivo ® | HC | QVQLVESCGGGGGGYMTRSAMGGGG (15) | YMTRSAMG (1) | Y |
| KO3-KLK6s- | LC | EIVLTGRSCGGFGFGYMTRSAMGGGG (17) | YMTRSAMG (1) | Y |
| Opdivo ® | HC | QVQLVESCGGFGFGYMTRSAMGGGG (18) | YMTRSAMG (1) | Y |

### EXAMPLE 7

### In Vivo Testing of Pro-Antibody Activation

In this example, FR1 peptide-KLK6s fused Avastin-KLK6s-Leptin was injected into BALB/c mice through i.v. injection. The serum samples and brain samples were harvested at 0.08, 2, 4, and 8 hours post-injection, respectively. The active antibodies and total antibody concentrations were determined by ELISA.

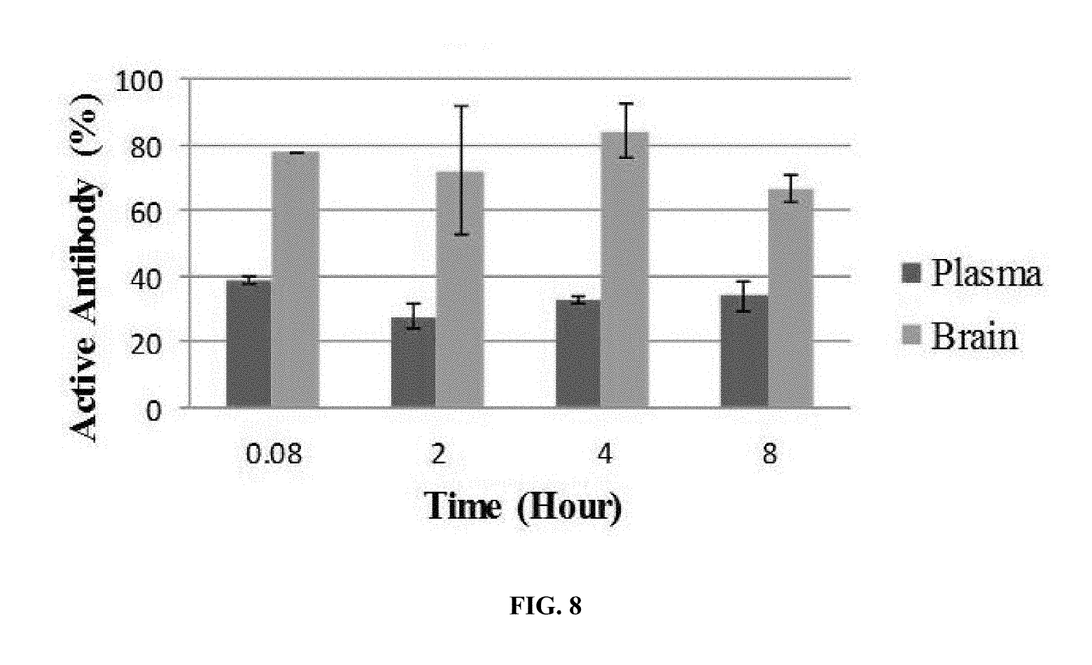

FIG. 8 shows results of in vivo antibody activation of the KLK6 substrate (KLK6s)-containing peptide-fused antibody in mice. In the plasma, the percentage of active FR1 peptide-KLK6s fused Avastin-KLK6s-Leptin was maintained at a reduced level, whereas the FR1 peptide-KLK6s fused Avastin-KLK6s-Leptin in the brain showed increasing percentages of the active antibody. These results show that the blocker-KLK6s-fused antibody can be reactivated in vivo in the CNS.

Some embodiments of the invention relate to methods for treating a CNS disease. A method in accordance with one embodiment of the invention comprises administering to a subject in need thereof an effective amount of a pro-antibody of the invention. The pro-antibody comprises a blocking peptide attached to the N-terminus of the heavy chain and/or light chain of an antibody or a binding fragment thereof. The binding fragment of an antibody may be an Fab, scFv, or F(ab′)₂, etc.

While the invention has been described with respect to a limited number of embodiments, those skilled in the art, having benefit of this disclosure, will appreciate that other embodiments can be devised which do not depart from the scope of the invention as disclosed herein. For example, other antibodies may be used instead of the ones shown in these examples. Similarly, the blocking peptide fragment can include any proteins or peptides linked by a KLK6 substrate sequence to the antibodies. Accordingly, the scope of the invention should be limited only by the attached claims.

What is claimed is:

1. An antibody prodrug capable of being selectively activated in a central nervous system (CNS) by protease KLK6, the antibody prodrug comprising:
an antibody, or a binding fragment thereof, for treating a disease or disorder in the CNS;
a KLK6 cleavable peptide fused to an N-terminus of a heavy chain and/or a light chain of the antibody; and
a blocker fused to an N-terminus of the KLK6 cleavable peptide.

2. The antibody prodrug according to claim 1, wherein the blocker comprises a protein, protein domain, or a peptide.

3. The antibody prodrug according to claim 1, wherein the KLK6 cleavable peptide is derived from a natural KLK6 substrate, a peptide phage library, a synthetic peptide library, or a peptide sequence that can be cleaved by KLK6.

4. The antibody prodrug according to claim 1, wherein the antibody is a monoclonal antibody, a bispecific antibody, a multi-specific antibody, or an antibody drug conjugate (ADC).

5. The antibody prodrug according to claim 4, wherein the antibody drug conjugate is an immunocytokine, an immunohormone, or an immunotoxin.

6. The antibody prodrug according to claim 1, wherein the antibody is for use in an application involving chimeric antigen receptor (CAR)-T cell.

7. The antibody prodrug according to claim 1, wherein the disease or disorder is a cancer, inflammatory disease, autoimmune disease, infectious disease, or neuron degeneration disease.

8. The antibody prodrug according to claim 1, wherein the antibody is a murine antibody, chimeric antibody, humanized antibody, or human antibody.

9. The antibody prodrug according to claim 1, wherein the antibody is anti-VEGF antibody or anti-PD-1 antibody.

10. A method for treating a disease or disorder in CNS, comprising: administering to a subject in need thereof an effective amount of the antibody prodrug according to claim 1.

11. The method according to claim 10, wherein the blocker comprises a protein, protein domain, or a peptide.

12. The method according to claim 10, wherein the KLK6 cleavable peptide is derived from a natural KLK6 substrate, a peptide phage library, a synthetic peptide library, or a peptide sequence that can be cleaved by KLK6.

13. The method according to claim 10, wherein the antibody is a monoclonal antibody, a bispecific antibody, a multi-specific antibody, or an antibody drug conjugate (ADC).

14. The method according to claim 13, wherein the antibody drug conjugate is an immunocytokine, an immunohormone, or an immunotoxin.

15. The method according to claim 10, wherein the antibody is for use in an application involving chimeric antigen receptor (CAR)-T cell.

16. The method according to claim 10, wherein the disease or disorder is a cancer, inflammatory disease, autoimmune disease, infectious disease, or neuron degeneration disease.

17. The method according to claim 10, wherein the antibody is a murine antibody, chimeric antibody, humanized antibody, or human antibody.

18. The method according to claim 10, wherein the antibody is anti-VEGF antibody or anti-PD-1 antibody.


SEQUENCE LISTING (18 sequences)

SEQ ID NO: 1 - protein, 8 aa, Artificial Sequence
  Synthetic
YMTRSAMG

SEQ ID NO: 2 - protein, 26 aa, Artificial Sequence
  Synthetic
DIQMGRQSCGGFGFGYMTRSAMGGGG

SEQ ID NO: 3 - protein, 25 aa, Artificial Sequence
  Synthetic
EVQLVESCGGFGFGYMTRSAMGGGG

SEQ ID NO: 4 - protein, 26 aa, Artificial Sequence
  Synthetic
DIQMGRQSCGGGGGGTAFRSAYGGGG

SEQ ID NO: 5 - protein, 25 aa, Artificial Sequence
  Synthetic
EVQLVESCGGGGGGTAFRSAYGGGG

SEQ ID NO: 6 - protein, 26 aa, Artificial Sequence
  Synthetic
DIQMGRQSCGGFGFGTAFRSAYGGGG

SEQ ID NO: 7 - protein, 25 aa, Artificial Sequence
  Synthetic
EVQLVESCGGFGFGTAFRSAYGGGG

SEQ ID NO: 8 - protein, 26 aa, Artificial Sequence
  Synthetic
DIQMGRQSCGGGGGGSSYISNYGGGG

SEQ ID NO: 9 - protein, 25 aa, Artificial Sequence
  Synthetic
EVQLVESCGGGGGGSSYISNYGGGG

SEQ ID NO: 10 - protein, 26 aa, Artificial Sequence
  Synthetic
DIQMGRQSCGGFGFGSSYISNYGGGG

SEQ ID NO: 11 - protein, 25 aa, Artificial Sequence
  Synthetic
EVQLVESCGGFGFGSSYISNYGGGG

SEQ ID NO: 12 - protein, 8 aa, Artificial Sequence
  Synthetic
TAFRSAYG

SEQ ID NO: 13 - protein, 8 aa, Artificial Sequence
  Synthetic
SSYISNYG

SEQ ID NO: 14 - protein, 25 aa, Artificial Sequence
  Synthetic
EIVLTQSCGGGGGGYMTRSAMGGGG

SEQ ID NO: 15 - protein, 25 aa, Artificial Sequence
  Synthetic
QVQLVESCGGGGGGYMTRSAMGGGG

SEQ ID NO: 16 - protein, 26 aa, Artificial Sequence
  Synthetic
EIVLTGRSCGGGGGGYMTRSAMGGGG

SEQ ID NO: 17 - protein, 26 aa, Artificial Sequence
  Synthetic
EIVLTGRSCGGFGFGYMTRSAMGGGG

SEQ ID NO: 18 - protein, 25 aa, Artificial Sequence
  Synthetic
QVQLVESCGGFGFGYMTRSAMGGGG